(19) United States
(12) Patent Application Publication
(10) Pub. No.: US 2022/0054603 A1
(43) Pub. Date: Feb. 24, 2022

(54) STABILIZED LIQUID ENZYME SUPPLEMENT AND USES THEREOF

(71) Applicants: Bernard W. Downs, Lederach, PA (US); Steven M. Kushner, Oldsmar, FL (US)

(72) Inventors: Bernard W. Downs, Lederach, PA (US); Steven M. Kushner, Oldsmar, FL (US)

(21) Appl. No.: 17/223,671

(22) Filed: Apr. 6, 2021

Related U.S. Application Data
(60) Provisional application No. 63/005,740, filed on Apr. 6, 2020.

(51) Int. Cl. A61K 38/48; A61K 9/127; A61K 47/24; A61K 9/00; A61P 3/00; A61K 38/47; A61K 38/46; A61K 33/00; A61K 33/06; A61K 33/04; A61K 33/22; A61K 33/42; A61K 33/32; A61K 33/30; A61K 33/18; A61K 33/24; A61K 33/34

(52) U.S. Cl. CPC A61K 38/4806; A61K 33/34; A61K 47/24; A61K 9/0053; A61P 3/00; A61K 38/47; A61K 38/465; A61K 33/00; A61K 33/06; A61K 33/04; A61K 33/22; A61K 33/42; A61K 33/32; A61K 33/30; A61K 33/18; A61K 33/24; A61K 9/127

(57) ABSTRACT

An enzyme containing composition is provided comprising a stabilized liquid enzyme supplement encapsulated in phospholipid structures. The enzyme supplements may be administered to humans so that the enzymes are rapidly absorbed into the blood and tissues in order to improve metabolism, immune competence, sleep quality, and aid digestion, in addition to general health, well-being, and overall quality of life.

### TECHNICAL FIELD

A stabilized liquid enzyme supplement is provided which is encapsulated in phospholipid structures. The enzyme supplements may be administered to humans so that the enzymes are rapidly absorbed into the blood and tissues in order to improve metabolism, immune competence, sleep quality, and aid digestion.

### BACKGROUND

It is well-known that phospholipids are important molecules in biological systems. Cells are surrounded by a layer of phospholipids called the phospholipid bilayer (generally, “lipid bilayer”). This layer makes up your cellular and intracellular organelle membranes, forming a selectively permeable barrier, and is critical to a cell's ability to function. Phospholipids are arranged so that their water-repelling (hydrophobic) or “fat-loving” tails are pointing inwards and their water-attracting (hydrophilic) heads are pointing outwards in this bilayer structure. This arrangement allows plasma membranes to be selectively permeable to dissolved substances such as proteins, ions, and water. In biological systems, phospholipids allow cell membranes to be fluid. Their unique characteristics allow the cell membrane to be more malleable, taking different shapes and expanding or shrinking when necessary, such as when cells have to travel through very narrow capillaries in single file, one at a time. Phospholipids also can act as signaling molecules for receptors inside and outside of cell surfaces, facilitating communications between cells. They can be split to produce secondary messengers in cellular systems. As a secondary messenger, phospholipids can signal for leukocytes to migrate to a site of infection, and they can also inhibit nerve cells when necessary.

Important Functions of Phospholipids

(1) Act as building blocks of the biological cell membranes in virtually all organisms.

(2) Participate in the transduction of biological signals across cell membranes.

(3) Act as efficient store of energy as with triglycerides.

(4) Play an important role in the transport of fat between gut and liver in mammalian digestion.

(5) Serve as an important source of acetylcholine which is the most commonly occurring neurotransmitter substance occurring in mammals.

(6) Donate phosphorus for the formation of AMP, ADP and ATP, important molecules for the formation and recycling of cellular energy.

One of the outcomes of a healthy diet combined with healthy digestion is the formation of liposomes from phospholipids in the diet. Owing to the diminished quality of the standard American diet, and the consequential wide-spread decline of digestive competence, the formation of liposomes in the gastrointestinal (“GI”) tract has been significantly compromised and diminished. Without the aid of the liposome, many of the nutrients would not otherwise adequately penetrate the epithelial wall of the intestines for eventual uptake into the cells. Liposomes are safe and important for facilitating optimal absorption of valuable nutrients. For example, naturally occurring liposomes are present in human breast milk. See, e.g., M. M. Koerner, et al., Electrodynamics of lipid membrane interactions in the presence of zwitterionic buffers, 101 BIOPHYSICAL J. 362 (2011), incorporated by reference here in its entirety. Liposome structures are biodegradable and biocompatible (“body friendly”), enabling absorption through most tissues in the GI tract and alimentary tract, from the mouth to the colon. In addition to water-soluble vitamins, liposomes are beneficial for effective in situ delivery of fat-soluble vitamins, trace minerals, and naturally occurring phytonutrients, including flavonoids, terpenes, and saponins. See, e.g., B. C. Keller, Liposomes in nutrition, 12 TRENDS IN FOOD SCI. & TECH. 25 (2001), incorporated by reference herein in its entirety.

There are no known liquid, encapsulated, and stable enzyme preparations available. There are many multi-enzyme supplements (containing many of the same or similar ingredients) on the market. However, all of these supplements are in tablet or capsule form and are designed to be only taken with meals to have a direct impact on the food ingested. All of these supplements do not affect systemic foundational health or re-supply enzyme reserves as they are “used up” with the digestion of foodstuffs. Even if these tablet/capsule supplements were taken on an empty stomach, there is no method provided for absorption of these enzymes into the bloodstream with which to impact systemic health.

The absorption mechanism of this technology was first evaluated in the efficacy of oral administration of a novel and safe iron free VMP35 MNC encapsulated in a proprietary SK713 SLP phospholipid Prodosome technology on anemia and blood properties (i.e., hemoglobin, etc.) were assessed in human volunteers. See, e.g., B. W. Downs, et al., The effect of VMP35 supplement ingredients encapsulated in a novel Phospholipid Prodosome SK713 SLP nutrient delivery technology observed as a result of changes in properties of live human blood, 5 FUNCTIONAL FOODS IN HEALTH & DISEASE 292 (2015), incorporated by reference herein in its entirety. See also US Pat. Appl. Publ. No. 2017/0049701 to Kushner et al., incorporated by reference herein in its entirety.

If new methods of providing enzymes could be developed, this would represent a useful contribution to the art.

### SUMMARY OF THE INVENTION

In an embodiment, a method for delivering an enzyme supplement is described, comprising the steps of: providing a formulation comprising at least one enzyme encapsulated in a multilamellar clustoidal phospholipid vehicle, the multilamellar clustoidal phospholipid vehicle comprising: a solvent, phosphatidylcholine of at least 70% purity, and an ionic mineral composition; and orally administering the formulation to a human subject. In a preferred embodiment, the enzymes can include proteases, lipases, carbohydrases, and amylases.

### BRIEF DESCRIPTION OF THE DRAWINGS

FIG. 1 depicts, in one embodiment, serum cholesterol levels before and after 90 days of intervention with N-SORB or placebo in healthy human subjects. Mean±SEM (n=19 placebo; n=21 N-SORB).

FIG. 2 depicts, in one embodiment, serum AST levels before and after 90 days of intervention with N-SORB or placebo in healthy human subjects. Mean±SEM (n=19 placebo; n=21 N-SORB).

FIG. 3 depicts, in one embodiment, serum low-density lipoprotein (LDL) levels before and after 90 days intervention with N-SORB or placebo in healthy human subjects. Mean±SEM (n=19 placebo; n=21 N-SORB).

FIG. 4 depicts, in one embodiment, serum creatinine levels before and after 90 days of intervention with N-SORB or placebo in healthy human subjects. Mean±SEM (n=19 placebo; n=21 N-SORB).

### DETAILED DESCRIPTION

Enzymes are involved in virtually all biochemical processes. Food must be broken down by endogenous digestive enzymes so that the appropriate nutrients, including amino acids, fatty acids, cholesterol, carbohydrates, vitamins, and minerals, can be absorbed and utilized as biological “building” materials in the body. Endogenous digestive enzymes are required to digest foods and nutrients in the consumed food. In addition to digestive functions, enzymes are crucial for optimal metabolic function and immune health. Consequently, impairment in the function of digestive enzyme can adversely affect absorption of functional nutrients from food. Impaired digestion can promote indigestion that can lead to bloating, gas, disrupted gut health, and malnutrition, especially in older adults. Furthermore, an impaired digestive system can overburden and weaken metabolic activities and the immune response. Gut-associated lymphoid tissue (GALT) is a prominent part of mucosal-associated lymphoid tissue and an abundant reservoir of metabolic enzymes, which regulates almost 70% of the entire immune system. Moreover, about 80% of plasma cells (mainly immunoglobulin A-bearing cells) reside in the GALT. The enzyme-dependent GALT is crucial in the process of inflammatory responses. Nutrient and fluid absorption in the gastrointestinal tract require lymphatic networks to both regulate interstitial fluid balance and transport lipids. Physical stress, excessive workload, unplanned daily routine, lack of physical activities, unhealthy food habits, and/or advancing age can lead to a rapid decline of digestive enzyme levels. These events cause imbalances in gastrointestinal homeostasis, promoting a range of gastrointestinal distresses. Moreover, digestive impairment can lead to other changes including metabolic alterations and immune dysfunction. Per a United States National Institutes of Health report, about 60 to 70 million people are afflicted by diseases of the digestive system. Two major underlying causes of poor or incomplete digestion include advancing age-induced decline of digestive enzymes and microbiome imbalances (NIH, U.S. Dept of HHS, “Opportunities and challenges in digestive diseases research: recommendations of the Nat'l Commssn on Digest Dis.” Bethesda, Md.: NTH; 2009. NIH Publication 08-6514).

Exogenous digestive enzymes have exhibited their ability to effectively metabolize diverse food ingredients including amino acids, fats, and carbohydrates as well as to play a vital role in absorption of nutrients, thus improving gut health. Previous studies demonstrate that exogenous enzymes could improve digestive process by supplementing the endogenous digestive capacity. Digestive enzymes have been shown to have beneficial effects in a variety of digestive disorders including lactose intolerance, cystic fibrosis, celiac disease, and many others. Several supplements for digestive disorders are available to enhance and maintain digestive health. Thus, new, more efficacious enzyme containing supplements are needed to better serve the human population.

N-SORB® is a novel KD120 multienzyme complex (MEC) of metabolically activated enzymes encapsulated in an SK713 SLP (non-GMO soy lecithin phospholipid) absorption technology (Prodosome®) for rapid absorption directly into the bloodstream to restore systemic enzyme potential and overall metabolic efficiency. Thus, N-SORB® is a multiple enzyme composition comprising a stabilized liquid enzyme supplement.

N-SORB contains a range of protease, amylase, and lipase enzymes including glucoamylase and alpha-galactosidase. These enzymes have been engineered to be activated in pH ranging from 6.5 to 8.5 and, contrary to popular belief, it is directed to be taken in an empty stomach. Prodosome technology has been shown to facilitate rapid absorption of the encapsulated ingredients into the blood, increasing their bioavailability. Previous clinical studies have demonstrated that intervention with N-SORB improves gastrointestinal and neuroendocrine functions (Downs, et al., “Safety and efficacy of a novel KD120 MEC multi-enzyme complex (N-Sorb™) in human volunteers, FASEB J. (April 2017) 31:1b312).

KD120 MEC restores systemic enzyme pools thereby improving systemic metabolic parameters while simultaneously improving the GI system to more efficiently metabolize ingested foodstuffs so maximum nutrition can be obtained. It does so by being taken without food in the SK713 SLP technology so that the enzymes may be absorbed intact into the blood stream and bodily tissues.

KD120 MEC is easily and rapidly absorbed via the sublingual mucosa (and then again through the GI mucosal border), gaining rapid (and sustained) access to the blood, restoring metabolic efficiency including that of erythrocytes, leucocytes, and platelets. KD120 MEC is also beneficial as a daily supplement containing essential and naturally derived proteases, amylases, lipases, alpha-galactosidase, and glucoamylase. As a result of strengthening enzyme activity both digestive and systemic, KD120 MEC improves nutrient availability and utilization enhancing cellular metabolic functions including eliminative processes.

The Prodosome encapsulation technology enables the KD120 MEC to achieve rapid and prolonged absorption of its supplement ingredients, allowing a rapid spike of essential enzymes to enter the bloodstream where they positively impact the systemic metabolic function. These enzymes in their arrangement are able to restore enzyme reserves which both reduce digestive burden on the GI tract but also directly benefit systemic cellular physiology to effectively utilize oxygen, water and nutrients (i.e. metabolism). As needed, these enzymes can still be utilized for digestive functions in the gut if taken with food.

The Prodosome encapsulated KD120 MEC restores enzyme reserves, metabolic efficiency, improves sleep quality, and overall foundational health! No other enzyme supplement has been shown to accomplish these actions since these supplements are always used up in the gut. The key to KD120 MEC is not just taking it on an empty stomach, but the fact that the Prodosome spheres can transport intact enzymes into the bloodstream to effect healthy change.

In one embodiment, the multiple enzyme composition comprising a stabilized liquid enzyme supplement may be administered to a human subject orally. Oral intake of the supplement is carried out by holding and swishing in the mouth for a minimum of 30 seconds and then swallowing. The supplement may be held in the mouth for longer periods of time if desired.

As used herein, the term “clustoid(s),” alone or in combination with other terms, unless stated otherwise, refers to clusters of liposomal spheres.

As used herein, the term “multilamellar clustoidal,” alone or in combination with other terms, unless stated otherwise, refers to clusters of liposomal spheres within a liposomal sphere and clusters of those liposomal spheres within a liposomal sphere, etc., up to hundreds of concentric layers.

As used herein, the term “prodosome,” or Prodosome®, alone or in combination with other terms, unless stated otherwise, refers to the “energetically enhanced (EFIquence-treated) liposome that comprises the complex of multilamellar clustoidal liposomal structures.” Specifically, “prodosome” refers to electrolyte (ion)-impregnated phospholipid liposome complexes having multilamellar clustoidal liposomal structures. Prodosome® absorption technology provides non-genetically modified organism soy lecithin phospholipid structures to facilitate absorption beginning in the sublingual mucosa, reduce degradation in the stomach, and then facilitate absorption through the intestinal mucosa starting in the duodenum.

As used herein, the term “liposome,” alone or in combination with other terms, unless stated otherwise, refers to a vesicle composed of amphiphilic lipids arranged in a spherical bilayer or bilayers. Liposomes are unilamellar or multilamellar vesicles that have a membrane formed from a lipophilic material and an aqueous interior that contains the composition to be delivered. In order to cross intact mammalian skin, lipid vesicles must pass through a series of fine pores, each with a diameter less than 50 nanometers, under the influence of a suitable transdermal gradient. Therefore, it is desirable to use a liposome that is highly deformable and able to pass through such fine pores.

As used herein, the term “bioavailability,” alone or in combination with other terms, unless stated otherwise, refers to a measurement of that portion of an administered drug that reaches the circulatory system (e.g., blood, especially blood plasma) when a particular mode of administration is used to deliver the drug. Enhanced bioavailability refers to a particular mode of administration's ability to deliver nutrients, including oligonucleotides, nutraceutical particles, and drugs to the peripheral blood plasma of a subject tin need relative to another mode of administration. For example, when a non-parenteral mode of administration (e.g., an oral mode) is used to introduce the drug into a subject in need, the bioavailability for that mode of administration may be compared to a different mode of administration. Further, bioavailability correlates with therapeutic efficacy when a compound's therapeutic efficacy is related to the blood concentration achieved.

As used herein, N-Sorb, or N-SORB®, is a novel KD120 multienzyme complex (MEC) of metabolically activated enzymes composed of proteases, amylases, lipases, alpha-galactosidase, and glucoamylase from natural sources (see Table 5). These enzymes are encapsulated in a SK713 SLP (non-GMO soy lecithin phospholipid) absorption technology (Prodosome®), available from VNI, Inc. (Lederach, Pa.).

Prodosomes

While the liposome is naturally a zwitterionic molecular complex, the inclusion of the mineral ions in a similar proportion that exists in human blood, within every portion of the present complex of liposomal clustoidal spheres creates previously non-existent electrical properties of the liposomes (called “prodosomes”). Based on electrostatic properties, mineral ions incorporated into the water used for creating liposomes become part of the liposome structure itself; resembling the ionic properties that exist in human blood, for example. This enhances the ability of the liposomal transport sphere to transport and facilitate encapsulated nutrient absorption. This is in addition to encapsulating supplemental minerals of a nutrient formula containing one or more nutrient components within their liposomal spherical structures as a nutrient or nutritional payload.

In one embodiment, the solar-dried electrolyte source material being infused into the phospholipids is ionic in nature. This property infuses the ions into the manufactured liposomes and creates electrical/energetic/frequency properties of the phospholipid-based liposomal structures. The liposome (prodosome), in essence, becomes a dynamically charged compound, resembling more of a biological material, with similar ionic amounts as exist in human blood, with greater bio-functionality and potential for transport and delivery of nutrients, and contributing beneficial biological activities on their own.

Electrolytes are also important intracellular pH buffers. Following the depletion of intracellular electrolytes and exhaustion of other primary buffers, hemoglobin is expended to maintain intracellular pH. This changes not only the oxygen-carrying abilities of the hemoglobin but also polarity (negative ion concentration), and results in excessive red blood cell aggregation. Improvement in red blood cell morphology and plasma rheology, for example, are evidence of improvements in blood viscosity, negative ion concentration, pH, and blood functionality, i.e., oxygenating and hydrating properties.

With the liposome (prodosome) now infused and saturated with a comprehensive range of naturally occurring energetically active ions, there is a greater potential that the entire multilamellar clustoidal structure may act as a pH buffering agent for the tissues. It is likely that there is a re-balancing of pH in tissues where the liposome releases its payload as well as when the liposomal membranes sequentially begin to degrade and release their bioactive ions. This re-balancing of pH and restoration of optimal ionic properties will foster a more advantageous environment for nutrient utilization. As pH rebalances, healthy blood morphology, rheology, and hematology (i.e., viscosity, form, structure, oxygenation, hydration, etc.) are restored.

Phospholipids have an adhesive property owing to the hydrophilic and hydrophobic properties of the molecule. As a result, a natural tendency of a phosphatidyl choline-based liposome is its ability to adhere to tissues, especially the mucosa of the GI tract. This attribute promotes transmucosal nutrient transport from the sublingual tissues in the mouth to the tissues of the intestine. Prolonged adherence of the liposome to the surface of the villi and microvilli translates to a longer portion of time that nutrients can diffuse across the membranes into the blood stream. More importantly, the extended time that the liposome remains attached to the mucosal membrane gives additional time for the mineral ions to saturate the same membranes. Moreover, the lipid bilayer construction created by phospholipids is readily incorporated into the cell membrane phospholipid bilayer. By continually saturating the junctions where nutrients are absorbed, an advantage is afforded for more complete nutrient transport. This is due to mineral ions' contribution to maintaining the osmotic gradient in the lipid bilayer of cell membranes that facilitate nutrient diffusion and maintain electroneutrality.

The fact that the liposome remains attached to the mucous membrane for longer periods of time means the mineral ions remain there as well. Again, this means that there is a longer period of time where nutrient exchange, facilitated by cellular ions, can be carried out. Moreover, the rationale of infusing the mineral ions within the entirety of the liposome is borne out by the fact that as each layer of the multi-lamellar sphere degrades and releases its nutrients in the GI mucosa, there is a simultaneous and consistent release of mineral ions as a result of liposomal (prodosome) degradation. This is as opposed to bound minerals just being present within the sphere of a “simple” liposome that can release at a single instant and then must be absorbed into the bloodstream. In an embodiment, the process of the present disclosure enables mineral ions to be available throughout the entire process where each successive layer of phospholipids and their nutrient contents, both fat and water-soluble, are being released from the disintegrating spheres, along with phospholipid-infused ions, and made available for diffusion and bioactivity.

Without being bound by theory, in an embodiment, the present invention contemplates the ability to increase Zeta Potential within the liposome (prodosome) itself and consequently in surrounding fluids where the prodosome degrades and releases its nutritional payload, including its free ions. As used herein, the term “Zeta Potential,” alone or in combination with other terms, unless otherwise stated, refers to the electrical potential of dispersed particles in colloidal solutions. The higher the Zeta Potential, the greater the dispersion and subsequent stability of the solution. A higher Zeta Potential, necessitating the term ‘Prodosome’ to replace the term liposome, indicates a stronger level of electrostatic repulsion within the solution, and consequently, a more stable liposome, which is understood to be a key factor in maintaining the biologically active properties and efficacy of a nutritional compound or nutrient formula. This not only holds true for the solution (i.e., the liposomal/prodosomal concentrate), but this potential can also be transferred to the surrounding tissues as the prodosome disintegrates/degrades. The electrostatic repulsion and separation of biological materials (i.e., erythrocytes, leukocytes, platelets, etc.) is exactly the environment that is desirable within the bloodstream of a human subject, for example.

Without being bound by theory, in an embodiment, the electrostatic repulsion and separation of biological materials (i.e., erythrocytes, leukocytes, platelets, etc.) helps to ensure adequate red blood cell circulation and, therefore, oxygenation, over a larger surface area. The opposite consequence (i.e., inadequate red blood cell circulation and oxygenation over a given surface area) would be aggregation and less free-flowing red blood cells. Accordingly, in an aspect, the present invention includes, but is not limited to, the creating of a transfer of Zeta Potential through the direction action of the prodosome itself as it enters into surrounding plasma. As the Zeta Potential increases in the surrounding blood, allowing for better circulation of red blood cells, the overall rheology of the blood is improved, thereby allowing for a greater flow of the nutrient payload that has been delivered by the multilamellar clustoidal liposome structures. Research has shown that both Zeta Potential and particle size within a colloidal solution can be modified by the inclusion of an ionic species, for example, according to embodiments of the presently disclosed clustoidal multilamellar SLP structures (prodosomes).

Without being bound by theory, recent research has also shown that varying degrees of vortex speed can decrease particle size in a colloidal solution while simultaneously increasing Zeta Potential, while also serving to allow embodiments of the present invention to increase surface area coverage. Embodiments of the present invention can increase surface area coverage through the use of high-speed RPMs within small mixing containers, allowing the mineral ions to more thoroughly disperse in a more uniform manner within the phospholipid matrix, which directly leads to a higher Zeta Potential. Moreover, typical Zeta Potential has to do with an electrokinetic potential between the surface of the colloidal particle and any point in the mass of the liquid medium. Without being bound by theory, it is expected that because embodiments of the present invention involve increasing ionic concentrations within the water prior to the production of the multi-lamellar liposome, multiple surfaces are generated, surrounded by multiple liquid mediums, into which the active substrate can permeate the inter-phospholipid molecular spaces or interstitial lumens. Thereby, in embodiments of the present invention, the multi-lamellar SLP structure is produced. Additionally, according to embodiments of the present invention, a Zeta Potential has been created within the multitude of layers of a clustered multi-lamellar liposomal sphere (i.e., prodosome). Consequently, in embodiments of the present invention, as the prodosome dissolves sequentially layer by layer, positive benefits of the increased Zeta Potential from each surface layer is conferred into the surrounding medium into which the prodosome dissolves, specifically, the sublingual mucosa (alimentary) and small intestine (GI), facilitating rapid and prolonged absorption into the bloodstream.

The absorption of food and most supplemental minerals primarily takes place within the small intestines, although ionic minerals can be absorbed through the sublingual mucosa. As food matter passes through the intestines, minerals transfer into the blood stream through the walls of the intestines by way of the villi. Without being bound by theory, minerals transferring into the blood stream by way of the intestinal villi can only happen if the minerals are in an ionic form. When the stomach is functioning properly, stomach acid normally ionizes minerals in foods and supplements. However, according to statistics, properly functioning stomachs are not commonplace in North America. Most mineral supplements contain bonded minerals (e.g., calcium carbonate, magnesium oxide, etc.) that must be ionized for optimal absorption and utilization in the body.

In one aspect of the present invention, encapsulating nutritional, nutraceutical, or pharmaceutical substrate(s) in soy lecithin phospholipid (“SLP”) structures enables superior absorption of nutritionally and pharmacologically therapeutic substances and/or enzymes. The present disclosure offers significant therapeutic health benefits due to its energetically enhanced phospholipid properties impacting delivery of certain enzymes, nutrients and/or drugs, including, but not limited to: (1) neuroprotection, regulation of brain activity, improved memory and resistance to stress, reduced depression risk, and mitigation of the progression of neurodegenerative diseases like ALS, MS, Alzheimer's disease, and Parkinson's disease; (2) positive influences on cellular growth, development, and energy generation due to participation in molecular transport, and cellular organelle and intracellular organelle structure and function; (3) acceleration of tissue and organism regeneration after trauma, damage, illness, and/or physical exertion, including wound healing; (4) limiting cholesterol absorption from the gastrointestinal tract; (5) beneficial outcomes in liver therapy (e.g., steatosis, alcohol intoxication, etc.); (6) inhibition of inflammation factors, some of which are pathogens of the alimentary canal and cancer promoters (e.g., of colon and adenoma); and (7) immune support. See, e.g., B. C. Keller, 2001.

More specifically, in certain embodiments, the present disclosure is directed to a clustoidal multi-lamellar phospholipid based material (“prodosome”) that is infused and fortified with an electrolyte mineral complex including more than 70 naturally occurring macro- and trace minerals in ionic form. As used herein, the term “trace minerals,” alone or in combination with other terms, unless stated otherwise, refers to naturally occurring minerals derived from, for example, evaporated inland sea water in an ionic form. Trace minerals, include, but are not limited to, iron ion, copper ion, zinc ion, manganese ion, selenium ion, chromium ion, iodine ion, and boron ion. Macro minerals include, but are not limited to, calcium ion, magnesium ion, phosphorous ion, potassium ion, chloride ion, and sulfur ion. In embodiments of the present invention, the final material possesses an electrical potential structurally integrated into the SLP sphere at a micron/nano level. The SLP sphere, according to embodiments of the present invention, has now become more than just a transport compartment, but also possesses its own unexpected beneficial functionality facilitating improved utilization of nutrients encapsulated within the SLP prodosomal spheres. In certain embodiments, the present invention provides a myriad of electrolytic materials, simultaneously with encapsulated nutrients, that contribute to and govern cellular fluid balance and, therefore, are instrumental in all metabolic processes including cellular exchange of nutrients and waste removal.

Existing liposome technologies use mostly lecithin and, especially those of higher quality, phosphatidyl choline. Regardless of the phospholipid source material, existing technologies are generally mixed in a stereotypical fashion with no other additives or compounds utilized within the source material(s). The result is generally a relatively unstable product that degrades of its own accord in a relatively short period of time due to variations in: temperature; agitation; composition of the substrate; interaction of the phospholipids with the encapsulated substrate; and pH; among other factors. The relative instability results in agglomeration leading to degradation and delineation of the phospholipid bilayer membrane of the multilamellar spheres.

Biological Capacitor

In embodiments of the present invention, the prodosome technology as described herein creates clusters of multilamellar prodosomal structures in concentric layers of activated ion-infused liposomes within a liposome; and the multilamellar clusters of those molecules within an activated ion-infused liposome; up to hundreds of concentric layers, described as multilamellar liposomal clustoids, referred to herein as SK713 SLP Prodosomes, or “Prodosomes.” In addition to protecting the nutritional contents, complex multilamellar clustoidal structures (i.e., the “SK713 SLP” complex) effectively function as biological capacitors, containing and confining the biochemical and/or energetic potential of the ion-infused (energy frequency imprinting) phospholipids. However, this biological capacitor function would not occur in normal liposomes (see, e.g., Table 1) and is only possible because of the energy frequency imprinting (also known as “EFIquence” technology, available from Victory Nutrition International, Lederach, Pa., United States) of the SK713 SLP process technology.

One objective, in an embodiment of the present invention, is to supply already naturally ionized minerals that can be fully absorbed in vivo. The Energy Frequency Imprinting (trading as EFIquence™ Technology) process infuses and saturates the phospholipids with a full spectrum of solar-dried ionic minerals from ancient sea beds that supply minerals in biocompatible amounts and a proportion to the blood.

The electrolytes within the phospholipid matrix, according to embodiments of the present invention, are in ionic form, in which the electrolytes are in the most natural state, in which they are naturally charged, biologically active minerals that are bioavailable and soluble in water. This material is derived from the Great Salt Lake, then solar-dried, and contains over 72 ionic minerals that are about eight to about ten times more concentrated than regular seawater and significantly more concentrated than colloidal minerals. Colloidal minerals are of larger particles size, and contain no ionic charge, as compared to the trace minerals used in embodiments of the present invention. Additionally, the ions contained in the Prodosomes are at a similar percentage volume to that which is present in healthy human blood.

As described herein, in embodiments of the present invention, the biological capacitor function of the multilamellar SLP clustoids (Table 2) would not occur in normal liposomes (Table 1) and is only possible because of the energy frequency imprinting (also known as “EFIquence™” technology, available from Victory Nutritional International, Lederach, Pa., United States) of the SK713 SLP process technology. The novel multilamellar clustoidal phospholipid encapsulation technology of the present invention (SK713 SLP/“Prodosomes”) was developed to facilitate more stable, competent, and comprehensive synchronized absorption and synchronized bioavailability and bioactivity of orally ingested nutrition. The SK713 SLP is distinctly unique and superior to any previous liposomal technologies and, unlike previous versions: contains more phospholipid substrate, which is impregnated and saturated with solar dried electrolytes in an ionic state; is demonstrably and significantly more stable; and is consistently more uniform and shown to be more efficacious for nutrient delivery than other liposomal technologies tested. Moreover, the ion-infused SK713 SLP makes a nutritional contribution to improving the structure and function of inter- and intracellular membranes and molecules.

TABLE 1
Electrical Resistance of Normal Liposome Solutions (Reference)

| Sample #1 Pure Liposome | 1000× Setting (in Ohms) |
|---|---|
| Distilled Water = 50 ml. | 600 |
| Drops 1 | 400 |
| Drops 2 | 380 |
| Drops 3 | 380 |
| Drops 4 | 360 |
| Drops 5 | 350 |
| Drops 6 | 300 |
| Drops 7 | 300 |
| Drops 8 | 300 |
| Drops 9 | 300 |
| Drops 10 | 280 |
| Drops 11 | 280 |
| Drops 12 | 280 |
| Drops 13 | 280 |
| Drops 14 | 280 |
| Drops 15 | 280 |
| Drops 16 | 280 |
| Drops 17 | 280 |
| Drops 18 | 280 |
| Drops 19 | 260 |
| Drops 20 | 260 |
| Drops 21 | 260 |
| Drops 22 | 260 |
| Drops 23 | 260 |
| Drops 24 | 240 |
| Drops 25 | 240 |
| Drops 26 | 220 |
| Drops 27 | 220 |
| Drops 28 | 220 |
| Drops 29 | 220 |
| Drops 30 | 200 |
| Drops 40 | 160 |
| Drops 50 | 150 |
| Drops 60 | 150 |
| Drops 70 | 140 |
| Drops 80 | 135 |
| Drops 90 | 125 |
| Drops 100 | 115 |

TABLE 2
Biological Capacitor Function of the Multilamellar SLP Clustoids

| Sample #2 | | | | |
|---|---|---|---|---|
| Prodosome | 1000× | 100× | 1000× | 100× |
| (Multi-lamellar | Setting | Setting (in | Setting | Setting (in |
| SLP) | (in Ohms) | Ohms) | (in Ohms) | Ohms) |
| Distilled | 600 | | 600 | |
| Water = 50 mL | | | | |
| Drops 1 | 350 | | 350 | |
| Drops 2 | 220 | | 220 | |
| Drops 3 | 160 | | 160 | |
| Drops 4 | 140 | | 150 | |
| Drops 5 | 140 | | 140 | |
| Drops 6 | 120 | | 120 | |
| Drops 7 | 120 | | 120 | |
| Drops 8 | 120 | | 100 | |
| Drops 9 | 100 | | 90 | |
| Drops 10 | 90 | | 80 | |
| Drops 11 | 80 | | 80 | |
| Drops 12 | 80 | | 70 | |
| Drops 13 | 70 | | 66 | |
| Drops 14 | 70 | | 64 | |
| Drops 15 | 65 | | 62 | |
| Drops 16 | 65 | | 58 | |
| Drops 17 | 60 | | 56 | |
| Drops 18 | 60 | | 56 | |
| Drops 19 | 56 | | 54 | |
| Drops 20 | 54 | | 54 | |
| Drops 21 | 54 | | 52 | |
| Drops 22 | 52 | | 50 | |
| Drops 23 | 50 | | 50 | |
| Drops 24 | 50 | | 48 | |
| Drops 25 | 50 | | 48 | |
| Drops 26 | 48 | | 48 | |
| Drops 27 | 48 | | 46 | |
| Drops 28 | 46 | | 44 | |
| Drops 29 | 46 | | 44 | |
| Drops 30 | 45 | 300 | 42 | 280 |
| Drops 40 | 38 | 220 | 32 | 200 |
| Drops 50 | 28 | 180 | 28 | 160 |
| Drops 60 | 28 | 170 | 26 | 150 |
| Drops 70 | 26 | 150 | 26 | 140 |
| Drops 80 | 24 | 140 | 24 | 130 |
| Drops 90 | 23 | 125 | 24 | 120 |
| Drops 100 | 22 | 120 | 22 | 120 |

As shown in the comparison of Tables 1 and 2, testing was performed to determine the difference in electrical resistance between distilled water, a basic liposome dissolved in distilled water, and the SK713 SLP Prodosomes dissolved in distilled water, and the trace mineral concentrate in pure form that is used in the processing of the prodosomes. A standard multi-meter (Armaco Brand 20A) was used and was set to measure ohms with a digital output. Ohms are a measurement of the electrical resistance that can be found in a particular solution or compound. Tests were run on both the 100× and 1000× setting, the 1000× setting being more sensitive to ionization. Pure distilled water was used as a control, and as the medium for dissolving the various liquids to be tested. All materials including the distilled water were allowed to reach room temperature. The amount of water used in each test was a volume of 50 mL and all came from a single bottle. All containers used for testing were glass. In all cases, each material to be tested was added 1 drop at a time into the water and the multimeter was used to detect resistance as determined by Ohm readings. After the initial tests were completed, identical testing was repeated to ensure uniformity of results.

In measuring pure distilled water, the detection of ohms, as shown on the digital readout, at the 100× setting, was not detectable, indicating infinite resistance and therefore no conductivity. At the 1000× setting, the reading was 600 (Table 1).

Next, the basic liposome was added 1 drop at a time to 50 mL of distilled water, with an Ohm reading being taken after each drop was manually stirred in the water (Table 1). On the 100× setting, there was no evidence through the multimeter readings to show any Ohms, and therefore no electrical conductivity, even up to 100 drops of the liposome solution in the water medium, confirming the electroneutrality of the phospholipid molecules. At the 1000× setting, 1 drop lowered the resistance from the 600 level to 400, 2 drops only changed the reading to 380, the same with 3 drops, while 4 drops of liposome lowered only to 360, and 5 drops to 350. At 6-9 drops the reading was maintained at 300 Ohms (Table 1).

Next, the SK713 SLP Prodosome was added 1 drop at a time to 50 mL of distilled water, with an Ohm reading being taken after each drop was manually stirred in the water (Table 2). At the 1000× setting, 1 drop lowered the resistance from the 600 level to 350, 2 drops changed the reading to 220, the same with 3 drops, lowering the resistance level to 220. Four drops of Prodosome decreased the reading to 140, and stayed the same at 5 drops. At 6-8 drops, the reading was maintained at 120 while the 9th drop of Prodosome lowered the Ohms to 100. After the second drop of Prodosome was added to the water medium and tested, and as subsequent tests were performed, the decrease in resistance and concurrent increase in conductivity over the basic liposome was approximately 3 times as great. Additionally, in comparing the Ohms reading of the basic liposome to the Prodosome testing after each drop 10-30, the increase in conductivity of the Prodosome material was consistently 3-4 times more than the basic liposome. Also, with the basic liposome being added up to 100 drops in the water, there was no evidence of lessening resistance and therefore no conductivity at the 100× setting. On the other hand, the Prodosome test done with the 100× setting on the multimeter did begin to show a lessening of resistance according to Ohms at drop number 30 and continued to gradually decrease in resistance at testing of drops 30-100.

Finally, the pure trace mineral concentrate (“TMC”) used in the production of the Prodosome was added to the distilled water in an amount equivalent to that found in the same volume of Prodosome (Table 3). In other words, the TMS was added to a pre-measured quantity of water at a fraction of the total volume of Prodo some so as to ensure the amount of TMC would be the same as exists in the Prodosomes at each measurement, drop for drop, comparing the Prodosome to the TMC. In this test, the ionization was strong enough to only require the multimeter to be used at the 100× setting. At 1 drop of the TMC, the reading was 1 drop 500, 2 drops 280, 3 drops 300, 4 drops 160, 5 drops 140, 6 drops 120, and drops 7 and 8 at 100. The minerals contained in the TMC are listed in US 2017/0049701.

TABLE 3
Electrical Resistance of Solutions Containing Pure Ionic Trace Minerals

| Sample #1 Pure Ionic | 100× Setting |
|---|---|
| Trace Minerals | (in Ohms) |
| Drops 1 | 500 |
| Drops 2 | 280 |
| Drops 3 | 200 |
| Drops 4 | 160 |
| Drops 5 | 140 |
| Drops 6 | 120 |
| Drops 7 | 100 |
| Drops 8 | 100 |
| Drops 9 | 80 |
| Drops 10 | 80 |
| Drops 11 | 75 |
| Drops 12 | 75 |
| Drops 13 | 65 |
| Drops 14 | 65 |
| Drops 15 | 65 |
| Drops 16 | 65 |
| Drops 17 | 65 |
| Drops 18 | 65 |
| Drops 19 | 65 |
| Drops 20 | 60 |
| Drops 21 | 45 |
| Drops 22 | 40 |
| Drops 23 | 38 |

The readings of the multimeter in Ohms for the Prodosomes versus the TMC were consistently less by an order of magnitude (10×), drop for drop. Again, while there was an order of magnitude greater drop in resistance from the TMC, the concentration of ions in both the TMC/water mixture and the Prodosomes/water mixture was the same. This suggests strongly that the Prodosome material is acting as an effective insulator (i.e., “biological capacitor”), and is evidence of the electrical activity showing in the Prodosome material only coming from the ionic minerals contained on the outermost phospholipid layer of the Prodosome clustoidal sphere. Being neutral, the distilled water medium does not allow the Prodosome sphere to completely disintegrate, therefore the balance of the ionic material would be contained, or insulated, in the lower levels of the multi-lamellar clustoidal spheres. This would also promote the benefits of the conductivity supplied by the release of the infused ionic TMC to be sustained over an extended period of time, as each layer of the multi-lamellar clustoidal Prodosome sphere sequentially disintegrates in the more alkaline environments of the body (i.e., mouth, intestine, and possibly blood).

The biological capacitor function of the multilamellar SK713 SLP clustoids would not occur in normal liposomes (See Table 1) and is only possible because of the energy frequency imprinting (also known as “EFIquence” technology) of the SK713 SLP process technology. The novel multilamellar clustoidal phospholipid encapsulation of embodiments of the present invention (SK713 SLP/Prodosomes) was developed to facilitate more stable, competent, and comprehensive synchronized absorption, and synchronized bioavailability and bioactivity of orally ingested nutrition. The SK713 SLP is distinctly and surprisingly unique and superior to any previous liposomal technologies and, unlike previous versions: contains more phospholipid substrate, which is impregnated and saturated with solar dried electrolytes in an ionic state; is demonstrably and significantly more stable; and is consistently more uniform and shown to be more efficacious than other liposomal technologies tested.

Clustoidal Multilamellar SLP Encapsulated Nutraceutical Multivitamin Formulations (SK713 SLP Encapsulated VMP35 Multinutrient Complex).

In one embodiment of the present invention, the prodosomes based multivitamin formulation induced a beneficial effect on the properties of human blood by promoting rapid delivery of their nutritional contents to a human subject in vivo. In a particular embodiment, a novel clustoidal multilamellar soy-lecithin-phospholipid encapsulation formulation (“SK713 SLP Encapsulated VMP35 Multinutrient Complex” or “VMP35 MNC”), which includes, among other ingredients, vitamins, such as vitamins A, C, D3, E, B1, B2, B3, B6, and B12. The formulation was designed to be administered transmucosally. The components of VMP35 MNC Formulation are described in Table 4. However, the transmucosal route of administration of this formulation was not intended to be limiting. As understood by a person skilled in the art, the studied multivitamin formulation is also suitable for other routes of oral administration. Testing results showed that VMP35 MNC is a superior nutraceutical supplement that is able to effect positive changes in morphological, hematological, and rheological properties, and to overcome the limitations of those with various underlying digestive inefficiencies. See, e.g., Y. Shoji, et al., Nutraceutics and delivery systems, 12 J. DRUG TARGETING 385 (2004), incorporated by reference herein in its entirety.

TABLE 4
SK713 SLP Encapsulated VMP35 Multivitamin, Mineral &
Phytonutrient Formulation

| | Per | Unit of |
|---|---|---|
| INGREDIENT | Serving | Measure |
| R/O water | 26300 | mg |
| Vitamin A (Retinyl Palmitate) | 5000 | IU |
| Vitamin C (Ascorbic acid) | 60 | mg |
| Vitamin D3 (Cholecalciferol) | 0.025 | mg |
| Vitamin E (Alpha-tocopheryl Succinate) | 15 | IU |
| Vitamin B1 (Thiamin HCl) | 1.5 | mg |
| Vitamin B2 (Riboflavin) | 1.7 | mg |
| Vitamin B3 (Niacin) | 20 | mg |
| Vitamin B6 (Pyridoxine HCl) | 2 | mg |
| Folic acid | 400 | mcg |
| Vitamin B12 (Cyanocobalamin) | 5 | mcg |
| Biotin | 300 | mcg |
| Pantothenic acid (d-calcium pantothenate) | 10 | mg |
| Calcium lactate | 100 | mg |
| Iodine (potassium iodide) | 0.15 | mg |
| Magnesium citrate | 100 | mg |
| Zinc sulfate | 10 | mg |
| Sodium selenite | 0.07 | mg |
| Copper gluconate | 1 | mg |
| Manganese sulfate | 2 | mg |
| Chromium chloride | 0.12 | mg |
| Potassium citrate | 99 | mg |
| Choline bitartrate | 20 | mg |
| Inositol | 20 | mg |
| White pine cone extract | 5 | mg |
| BiAloe Concentrated 200:1 Water Extract | 20 | mg |
| VMP35 1:1 Herbal Blend: | 1700 | mg |
| Astragalus Root extract 1:1-247.5 mg | | |
| Ginger Root extract 1:1-99.95 mg | | |
| Green tea Leaf extract 1:1-199.92 mg | | |
| Fo ti Root extract 1:1-199.92 mg | | |
| Hawthorne berry extract 1:1-150.96 mg | | |
| Elderberry extract 1:1-99.95 mg | | |
| Eluthero Root extract 1:1-150.96 mg | | |
| Chamomile Flower extract 1:1-199.92 mg | | |
| Citrus bioflavonoids (from rose hips) 1:1-199.92 mg | | |
| Gotu kola Leaf extract 1:1-150.96 mg | | |
| SK713 SLP | 342 | mg |

One of the major components of VMP35 MNC formulation is a specially prepared high grade soy lecithin material that contains a minimum of 85% phosphatidylcholine (“>85PC”), an essential phospholipid, while most lecithin products contain only 19-21% PC. See, e.g., C. R. Scholfield, Composition of soybean lecithin, 58 J. AM. OIL CHEMISTS'SOC'Y 889 (1981), incorporated by reference herein in its entirety. The high PC content in SK713 SLP ensures thorough formation of liposomes. In addition to acting as biological capacitors and protecting the nutritional contents, multilamellar liposome phospholipids offer several health-related benefits. Due to their role in molecular transport, phospholipids also influence cell growth and development, and speed up organism regeneration after physical exertion. Phospholipids limit cholesterol absorption from the gastrointestinal tract and are beneficial in liver therapy, for instance, in the treatment of steatosis. Phospholipids inhibit inflammation factors, some of which are pathogens of the alimentary canal and promoters of cancers, for example adenoma, and colon cancer. See, e.g., A. Ambroziak, et al., Milk phospholipids as nutraceutic, 34 POLSKI MERKURIUSZ LEKARSKI 62 (2013), incorporated by reference herein in its entirety.

Clustoidal Multilamellar SLP Encapsulated Multienzyme Formulations (SK713 SLP Encapsulated KD120 Multienzyme Complex), i.e., N-SORB®.

The multi-lamellar or multisphered-multilayered-clustoidal structure of SK713 SLP, unlike standard liposome technology, is capable of encapsulating a diverse range of nutrients simultaneously, including enzymes selected from, but not limited to, proteases, amylases, and lipases (including glucoamylase and alpha-glycosidase). Through experimentation, SK713 SLP was found to form vesicles made up of hundreds of concentric lipid bilayers that range in size from 100 nanometers to 500 micrometers and are made up of a few dozen to several thousand molecules. See, e.g., B. C. Keller, Liposomes in nutrition, 12 TRENDS IN FOOD SCI. & TECH. 25 (2001), incorporated by reference herein in its entirety. As soon as the concentration of phospholipids reaches critical mass, the water-repelling ends organize to form the liposomes with the lipophilic (fat-attracting) hydrocarbon chains oriented inwards and the hydrophilic (water-attracting) groups facing outwards, forming the lipid bilayer structure.

In an embodiment, the enzyme ingredients include the following multi-enzyme formulation, in a blend available from National Enzyme Company, Forsyth, Mo., as shown in Table 5. The enzyme formulation is a broad-spectrum digestive enzyme blend formulated with enzymes selected from proteases, amylases, carbohydrases, and lipases.

TABLE 5

| Ingredient | Source | Activity/Dose | mg* |
|---|---|---|---|
| Amylase | Aspergillus oryzae | 10,309 DU | 68.7 |
| Protease¹ | Aspergillus oryzae | 54,260 HUT | 67.8 |
| Lipase | Rhizopus oryzae | 1,107 FCCLU | 51.3 |
| Protease² | Aspergillus oryzae | 24,010 HUT | 47.9 |
| Protease | Bacillus subtilis | 16,278 PC | 13.3 |
| Glucoamylase | Aspergillus niger | 13 AGU | 12.5 |
| Alpha-Galactosidase | Aspergillus niger | 54 GalU | 3.6 |
| Lipase | Aspergillus niger | 68 FCCLU | 3.3 |

*Daily value (DV) not established.
¹Metabolically activated at ca. pH 6.0-6.5
²Metabolically activated at ca. pH 8.0-8.5

The SK713 SLP multilamellar liposomes form spontaneously as the electrostatic and adsorptive properties lower surface tension (surfactant). The net result is thorough and complete phospholipid encapsulation (or entrapment) of nutritional ingredients within multiple layers of nano- to low micrometer-sized spheres. This electrostatic encapsulation is effective for encapsulating and transporting both water and fat-soluble nutritional ingredients including phytonutrients within the same spherical structure. See, e.g., A. Akbarzadeh, et al., Classification, preparation, and applications, 8 NANOSCALE RESEARCH LETTERS 102 (2013); W. Helfrich, Size distributions of vesicles: the role of the effective rigidity of membranes, 47 J. PHYSIQUE 321 (1986); each incorporated by reference herein.

a. Encapsulation of Nutrients

One of the limitations of encapsulating nutrients within the SLP transport spheres is the relative insolubility of some ingredients in water. Many nutritional compounds, especially inorganic minerals and resinous phytonutrients, are not readily soluble in water. To overcome this obstacle, prior to SK713 SLP processing, all materials are pre-processed in a low sheer tri-blender using jet-compression-particle-processing technology. This step is akin to a wet-milling process. In essence, the nutritional/nutraceutical materials are added directly to distilled water. The admixture is then blended at a low and consistent speed for a specific time, depending on the viscosity of the liquid and the physical and chemical properties of the added components. At the same time, water is circulated to create a secondary motion. No excess heat is produced in the mixing process. The low heat production combined with low shear used in the mixing step preserves the physicochemical stability of the nutrients and botanicals contained within the solution or suspension. The process continues for a period of time to substantially reduce particle size and to achieve consistency and uniformity of the mixed materials over successive batches. The electrolyte-impregnated SK713 SLP compound is then added to encapsulate these nutraceutical particles with greatly reduced particle size. Importantly, this preparation greatly improves bioavailability of the nutrients and botanicals. This preparation further ensures that previously insoluble materials can now be blended and dispersed into a semisolid or even a liquid state. The liquid concentrate is made up of the high-grade lecithin (>85% PC) combined with an amount of alcohol in exact proportions and blended at specific speeds for a specified time to achieve a solution with the right consistency, viscosity, and grade of material. The SK713 SLP material can then be blended into the liquid nutritional compound under precisely required speeds and blending times based on the material in the supplement as well as the batch size. The same process can be utilized for preparing topical formulation to achieve enhanced delivery. The amphipathic (hydrophilic and hydrophobic) properties of SK713 SLP allow it to encapsulate nutraceutical ingredients contained in a liquid medium and to serve as an efficient transmembrane delivery vehicle for these nutrients. The SK713 SLP delivery vehicles or spheres as set forth above comprise all natural generally recognized as safe (“GRAS”) ingredients or pharmaceutically/nutraceutically acceptable ingredients, which are suitable for human consumption.

b. Multilamellar Sphere Components

The SK713 SLP multilamellar spheres contain large quantities of electrolytes and hydroxyl-rich botanicals that contribute bioflavonoids and assist in maintaining healthy pH, proper hydration, and the transport and utilization of vital nutrients. The SK713 SL phospholipid spheres are zwitterions, methyl donors, and potential alkalizing buffer. See, e.g., G. Bouchard, et al., Theoretical and experimental exploration of the lipophilicity of zwitterionic drugs in the 1,2-dichloroethane/water system, 19 PHARM. RESEARCH 1150 (2002), incorporated by reference herein in its entirety. Zwitterions carry both positive and negative charges and may lower the energy requirement for transporting molecules thereby enhancing absorption by spreading the nutrient out over a larger surface area.

Zwitterions are soluble in many solvents, e.g., water. The SK713 SL phospholipid spheres have a natural “adhesive” property that enhances the ability of the body to absorb their nutritional contents. Specifically, in certain embodiments, the present invention relates to a novel soy-lecithin-phospholipid-nutrient encapsulation technology, which could achieve rapid onset and improved bioavailability of the nutrients encapsulated within clustoidal multilamellar soy lecithin (SK713 SLP) structures.

As set forth above, the presence of embedded free ions in SK713 (prodosome) enhances bio-electrical properties of the liposomal delivery system in an aqueous solution (See Table 2) and in the blood making it superior to conventional phospholipids in terms of its conductive properties and biological compatibility and functionality. Without being bound by any theory, it is expected that the loading of ions, enzymes, and/or other nutritional ingredients greatly increases the absorption of nutrients and promotes synergistic effectiveness of the simultaneously absorbed nutrients. The molecular structure created in the SK713 liposomal delivery system acts like a biological capacitor that can transport a variety of nutrients simultaneously across the sublingual mucosal membranes in the mouth and/or the wall of the small intestine into the portal circulation.

Application of Prodosome (SK713 SLP) Delivery System in Oral Administration

It is likely that the SK713 SLP spheres provide protection of the encapsulated nutritional contents within the multilamellar structures against the harsh acidic environment in the stomach. This protection enables the nutrients within the spheres to reach the small intestine intact, which promotes greater nutritional synergy in absorption and utilization. The entire SK713 SLP process helps to create a formulation that enables nutrients to disperse over a larger surface area within the small intestine. Initially, the low-sheer tri-blender jet compression technology decreases particle size of larger and more granular or resinous materials. The smaller particle size of a particular nutrient will allow this nutrient to cover a broader surface area once it reaches the small intestine. In addition, encapsulation within the SK713 SLP spheres can decrease particle size even further, especially of fat-soluble vitamins and phytonutrients. As the remaining mass of nutrients that does not absorb through the sublingual mucosa reaches the small intestine, it is likely to be absorbed through diffusion across the epithelial wall of the small intestine. The process of decreasing particulate size of these nutrients allows the entire mass of nutrients to disperse over a larger area of the small intestinal wall. This dispersion greatly increases the surface area into which nutrients can be absorbed so that less of the nutritional intake passes into the large intestine for elimination.

The multi-lamellar prodosome compositions and methods described above, the effect of prodosome-encapsulated VMP35 MNC on human blood may be further understood in connection with the following Examples. In addition, the following non-limiting examples are provided to illustrate the invention.

### Example 1

Method of Producing Clustoidal Multilamellar Soy Lecithin Phospholipid (SLP)

Step 1. Generally, a nutritional, nutraceutical, or pharmaceutical active ingredient substrate is processed through an advanced wet milling/particle compression process to facilitate a type of mechanical predigestion of substrate that enables more of the substrate to be encapsulated in the phospholipid spheres. Thoroughly wet milling the substrate significantly increases surface area of the substrate and enables a higher concentration and wider range of substrate ingredients to be homogenized and encapsulated in the Prodosome process.

The following steps are done in relatively small batches (approximately 5-gallon containers) to achieve an optimal speed ensuring the most complete and thorough homogenization of constituents. Following each step below, blending should be performed in small circular motions in the opposite direction of the rotation (counter-rotation) of the blender blade to increase the torsion to effect the interaction of ions with phospholipids over a greater fluid surface area and produce an energetically enhanced homogeneous mixture. Generally, start with an amount of water between 40-80% of total final volume. Heat water to a temperature between 90 degrees F. and 140 degrees F.

Step 2. In a 5-gallon stainless steel drum of water, solar evaporated mineral/trace mineral liquid concentrate between 1 to 120 g/kg of water was mixed in at a level ranging from 0.1% to 12.0%. this mixture was blended for a time between 1-5 minutes at a speed between 3,000 and 25,000 RPM in a high-RPM spinning vortex of water between 300 and 800 g/kg of total mixture to completely and uniformly disperse ions into what is now “structured water.” (Trace mineral liquid concentrate is available from Trace Minerals Research, Ogden, Utah, United States; see also FIG. 16 of US 2017/0049701).

Step 3. High-grade lecithin containing >85% Phosphatidylcholine (“PC”) at between 2 and 200 g/kg of the total mixture was added, corresponding to between 2% to 20%, respectively, and thoroughly mixed into the ion-rich water, blended between 1 and 5 minutes at a speed from 3,000 to 25,000 RPM, depending on substrate viscosity. Then, a small amount of ethyl alcohol was added (not less than 150 proof), at between 50 and 450 g/kg of the total mixture, and blending continued between 1 and 5 minutes at a speed of between 3,000 and 25,000 RPM depending on substrate viscosity. The mixture is then allowed to cool. As a result, the phospholipid structures are completely impregnated and saturated with free ions, achieving a completely homogeneous mixture of electrolytically “charged” SK713 SLP material. Variants of the procedure include: adding between 2 and 20% amounts of phosphatidyl choline with a PC content of no less than 70%. Adding between 5 and 45% USP Alcohol, at a level no less than 150 proof. The mixing procedure can include ultrasonic mixing.

Step 4. This mixture is then added to the nutritional, nutraceutical, or pharmaceutical active ingredient substrate of Step 1 in a blender and blended thoroughly to facilitate complete encapsulation of the substrate. In embodiments of the present invention, a level of between 0.5% and 10% can be used in “prodosoming” finished products depending on the composition and state (aqueous or dry) of the substrate being encapsulated.

The process may be varied slightly, within a narrow parameter, as to the degree of phosphatidyl choline (PC) content, depending on the end usage required. Limited variance of PC content of finished Prodosome may alter viscosity of liposomal material without creating any loss of advantage. Differing viscosity prodosomes may be required depending upon active ingredient intended for encapsulation, such as material more or less soluble, or materials containing higher levels of lipids. Trace mineral concentrate amounts can also be varied to some extent, depending on the substrate and benefit endpoints.

This mixing process evidently catalyzes association between electrolytes and other molecules within the total substrate (i.e., methyl and phosphoryl groups); certain B vitamins with methyl and/or phosphoryl ligands; also facilitating the permeation of substrate material into the phospholipid intermolecular spaces of the Prodosomes.

This process enables comprehensive and uniform encapsulation of nutritional and/or pharmaceutical ingredients in the SK713 SLP phospholipid prodosome capsules, facilitating superior absorption of nutritionally and pharmacologically active therapeutic substances that provide benefits following absorption of the energetically enhanced electrolyte-impregnated phospholipids.

The present disclosure includes specific materials with exacting levels of each, blended with distinct sequence and timing. The SK713 sphere is unique in many aspects, as follows.

A. Higher levels of PC-rich lecithin help to ensure stability and more comprehensive encapsulation.

B. Mixing of total compound in smaller containers, thereby allowing more thorough and uniform blending. This is as opposed to typical mixing on larger scales, which hampers proper fluidization.

C. Part of the total methodology of this invention requires pre-treatment of nutrients to be encapsulated. This can include, but is not limited to, wet milling, or partial dissolution using low or high shear wet milling (depending on substrate to be milled), to make active ingredients uniformly smaller and more accepting of the invention's encapsulation. This method also protects the integrity of the active compound being treated.

D. Other important reasons for mineralizing the water are decreasing Zeta Potential and improving stability. Typical water used in pharmaceutical/nutraceutical manufacturing is distilled through de-ionization or reverse osmosis. This form of water, while pure, typically has aggressive receptor properties vs. aggressive donor properties. As a “receptor,” it can become acidified by complexing with CO₂ (for example) as well. Empty, aggressive reception, and/or acidified water can disrupt surrounding mediums, including aqueous mediums containing nutrients. By aggressively mixing the water in a consistent vertical motion, the water becomes more structured. This motion also stabilizes the water portion of the liposomal sphere with added electrolytes, which causes the water to become more biocompatible, stable, and less disruptive to the nutrients contained therein. Therefore the entire final prodosome structure is more stable.

E. The invention starts with pharmaceutical grade water to ensure purity, and then adds a precise pre-measured amount of mineral electrolytes at the appropriate time to “mineralize” the water as just indicated above. This process ensures uniformity of mineral levels and distribution during each production process and also ensures a finished compound that has more of the biocompatible properties of body fluids and more readily promotes competent cell metabolism. Also, unlike relying on mineral water from a natural source, which can have impurities, varying potencies of minerals, and a complete absence of one or more mineral compounds, the process of the present invention ensures that the mineral electrolytes are supplied in uniform, ample, and comprehensive amounts. To this point, a 30-50 gallon batch of finished product was allowed to sit for 7 hours and experienced an exothermic reaction in which the temperature of the batched product rose up to 98.6 Fahrenheit, i.e., the temperature of body fluids, and then stopped. The present invention is creating a specific resonance that is completely biocompatible with body fluids.

F. The invention's inclusion of trace minerals contributes to intracellular pH regulation and homeostasis and pH stability in the liposomal sphere contained within the product prodosome, especially important because enveloped nutrients (e.g., Vitamin C) may disrupt pH balance. By avoiding this circumstance, additional stability is provided for the liposomal sphere contained within the product prodosome. Furthermore, the ability of the SK713 liposomal sphere contained within the product prodosome, infused and saturated with our special mineral rich electrolyte material, is that the sphere can impart, through the action of mineral buffering, a pH balancing effect within the bloodstream concurrently with the release of the contained nutrients. It should not be inferred that the pH of the SK713 or its substrate impose any buffering effects because of their pH properties. Rather, the SK713 and the ionic constituents contribute buffering potential as need for the body's homeostatic requirements. This phenomenon can improve cellular uptake and utilization of available nutrients.

Other known liposomal technologies are plagued with instability; gradual and continual degradation of liposomal capsules; and substrate “leakage” out of degrading and delineating liposomes ultimately results in a reduction and eventual loss of liposomal encapsulating benefits. Evidence of this degradation are visible in product containers as solid residues continue to amass, precipitate, and accumulate on the bottom of the containers. In contrast, thoroughly and completely “prodosomed” product remains completely and evenly dispersed and homogenized throughout the blended mixture. The SK713 process helps to ensure that capsule stability, homogeneity, and therefore stronger and more sustained benefits, occur from products treated with prodosomes in embodiments of the present invention.

Surface Tension Measurement

Other beneficial properties are evidenced by the Surface Tension testing done on standard liposomes vs. Prodosomes as prepared in Example 1. Testing was performed by NSL Analytical. Two liquid samples were submitted for Contact Angle measurement on a glass slide surface. The test outlined was performed on both samples. The measurements were recorded at five second intervals due to the small area of contact. Once the drop (10 μL) was in contact with the surface, the first measurement was recorded, and the second measurement was recorded after approximately five seconds, and the same for the third, fourth, and fifth. Sample #1 (standard liposome) demonstrated an average Contact Angle of 39. Sample #2 (Prodosome) demonstrated an average Contact Angle of 47.7. The inclusion and specific mixing process of the trace minerals into the Prodosomes increased the average level of surface tension by 22.3%. The increased surface tension has a direct and significant impact on liposomal integrity and can be attributed to the SK713 Process, which as previously discussed, increases Zeta Potential, thereby reducing agglomeration and increasing the dispersion and subsequent stability of the solution. A higher Zeta Potential leads to a stronger level of electrostatic repulsion within the solution and subsequent stronger liposomal shell(s) in the clustoidal multilamellar SLP prodosome structure of Example 1.

Advantages produced by this process include increased stability of the liposomal transport sphere contained within the product prodosome while simultaneously not adding to the cost or burden of producing the material. It also affords an increased opportunity to enhance cellular uptake of nutrients, both by balancing extracellular and intracellular pH and by bolstering extra—and intracellular fluid exchange. These actions occur concurrently with the delivery of nutrients, which creates additional synergies to benefit health. A replenishment of electrolytes is vital to maintaining a balanced osmotic gradient within plasma to ensure optimal oxygenation and correct hydration via maintaining optimum pH. It is this correct hydration and pH that affects all other usage of nutrients delivered by the liposomal-type sphere contained within the product prodosome.

In embodiments of the present invention, the process as described herein is focused on a new paradigm of altering the functionality of the liposome, giving it a dual purpose. With the SK713 Prodosome, the liposome now acts as both a delivery vehicle and a functional enhancer of the receptor or target of the delivered materials.

Other advantages also include low cost of production; ease of transport for usage on site; no additional or unusual equipment needed for usage; able to be stored at room temperature; better stability of SK713 material and better stability of liposomal material containing enveloped nutrients within the product prodosome; process uses pre-preparation of active ingredients to be Prodosomed in order to ensure better and more thorough encapsulation; and the creation of electrically charged, energy-enhanced phospholipids of the Prodosome that acts as a transport vehicle while also actively influencing cellular integrity for enhanced utilization of nutrients.

### Example 2

Preparation of KD120 Multi-Enzyme Complex (MEC) and Encapsulated Enzyme Composition (N-Sorb)

KD120 MEC is produced through a series of steps. First, the multi-enzyme complex is synthesized through fungal fermentation which is specifically designed to be active in a pH range of 6.0-8.5. See Table 5. Next, these enzymes in an amount of about 25% of the total volume (i.e., ca. 25 kg) are mixed in a base of vegetable glycerin to provide a total mixture of ca. 100 kg which offers no substrate with which the enzymes can react. Next, using a paddle blade mixer at a minimum of approx. 2000 rpm (but not exceeding about 3000 rpm in order to preserve the structure and activity of the enzymes employed) the enzyme/glycerin mixture is encapsulated into phospholipid rich ionically enhanced SK 713 SLP spheres mixed at high speed for ca. 15-20 min. to bond with/into the phospholipid architecture. The temperature of this process should be maintained below about 110° F. (again for preservation of the enzymes). This creates a one-of-a-kind fully stable liquid enzyme supplement.

### Example 3

Randomized, Double-Blind Placebo-Controlled Study

The objective of this study was to determine the safety and efficacy of N-SORB in 40 healthy male and female human subjects over a period of 90 consecutive days. Overall physical health, blood glucose, liver enzymes, lipid profile, red and white blood cell properties, serum cytokine levels, body weight, body fat mass, lean mass, android and gynoid fat, quality of life (QOL), energy and sleep pattern (Pittsburgh Sleep Quality Index [PSQI)), and digestive health were evaluated prior to and after 90 days of intervention.

Study Design

The study design, recruitment and methods were performed in compliance and accordance to the ICH guidelines for Good Clinical Practices, including the archiving of all relevant essential documents, and as per international standards guaranteed by the Declaration of Helsinki and its subsequent amendments. The University of Wyoming's Institutional Review Board (IRB) approval was obtained from this investigation (Protocol #20170118SN01429 Re: IRB Proposal “Pilot Clinical Study to Evaluate the effect of a Proprietary Enzyme Formulation on Cardiometabolk Parameters in Healthy Volunteers”). Informed consent was obtained from all subjects after discussing with them the study details and the extent of their individual involvement in the study. Participants were recruited through voluntary response to a flyer displayed throughout the community in Laramie, Wyo. (Albany County). The consent form was submitted with the protocol for review and approved by the IRB for this study. The formalized consent of a subject using the IRE-approved consent form was obtained before the subject could be included in any study procedure. Consent forms were signed by the subjects and the investigator-desig-nated research professional. Informed consent was obtained during the primary screening. Subjects also completed a brief health questionnaire. Patient confidentiality was strictly maintained. Adverse event monitoring was strictly enforced.

A total of 46 healthy volunteers (males=25; females=21; mean age: 25.8±12.1 years) participated in the study, while 6 subjects (males=4; females=2) dropped out. Thus, a total of 40 subjects completed the study. Subjects were randomly assigned, using a computer program, to receive either a placebo or N-SORB, 1 mL, twice daily, over a period of 90 consecutive days. N-SORB, a unique multi-enzyme formulation, is composed of proteases, amylases, and lipases (including glucoamylase and alpha-glycosidase) from natural sources. These enzymes are formulated in a non-genetically modified organism soy lecithin phospholipid absorption technology [Prodosome®] to facilitate absorption beginning in the sublingual mucosa, reduce degradation in the stomach, and then facilitate absorption through the intestinal mucosa starting in the duodenum. Both placebo (vehicle only) and N-SORB samples were prepared in a GMP-NSF certified facility and provided by VNI, Inc. All subjects were instructed to have a decent healthy lifestyle during this clinical investigation.

Subjects also completed a brief health questionnaire during this visit. In addition, anthropometric measurements were taken twice with the mean of the measurements used as the final reading. Height was measured after the removal of shoes using a stadiometer to the nearest millimeter. Body weight was measured using digital scales with heavy clothing such as jackets and coats removed. Body mass index (BMI) was calculated using the following formula: BMI=weight (kg)/height² (m²). Physical health was critically assessed pre- and post-intervention.

Multiple endpoints including blood glucose, liver enzymes, lipid profile, red and white blood cell properties, and body weight were assessed pre- and post-intervention.

A GE Lunar iDXA densitometer equipped with a software, version Encore 2005, 9.15.010 (GE Healthcare) was used to assess the body composition including lean mass, fat mass, tissue mass, android, gynoid, and bone mineral mass composition in both placebo- and N-SORB-supplemented subjects at the beginning and after 90 days intervention.

After overnight fasting, blood samples (2×10 mL each) were drawn at the Wyomed Laboratory (Laramie, Wyo.) into Vacutainer tubes and used for clinical chemistry. Extensive blood chemistry analyses including blood urea nitrogen (BUN), creatinine, alkaline phosphatase (ALP), aspartate aminotransferase (AST), alanine aminotransferase (ALT), blood glucose, total cholesterol, triglycerides, high-density lipoprotein cholesterol (HDL), low-density lipoprotein cholesterol (LDL) and very low-density lipoprotein cholesterol (VLDL), were performed in both placebo and N-SORB-treated subjects at the beginning and after 90 days of intervention. An additional aliquot of blood was used for cytokine analysis.

Serum cytokine levels including interferon gamma (INFγ), interleukin-2 (IL-2), interleukin-4 (IL-4), interleukin-6 (IL-6), interleukin-8 (IL-8), interleukin-10 (IL-10) and tumor necrosis factor-alpha (TNFα) were determined by using a Bio-Plex Pro Human Cytokine 8-plex assay at the beginning and following 90 days of intervention.

At the beginning of the study and following an interval of 90 days the subjects were asked to complete the Pittsburgh sleep quality index (“PSQI”) (Buysse D. J., et al., “The Pittsburgh sleep quality index: a new instrument for psychiatric practice and research,” Psychiatry Res. (1989) 28(2):193-213.

The subjects were asked to complete the World Health Organization Quality of Life (“QOL”) (WHOQOL-BREF) before and after intervention. The WHOQOL-BREF contains a total of 26 questions, rated on a 5-point Liker scale from 1 (never) to 5 (always) (The WHOQOL Group. Development of the World Health Organization WHOQOL-BREF quality of life assessment. Psychological Med. (1998) 28:551-558).

Total white blood cell count, red blood cell count, hemoglobin, hematocrit, platelets, mean corpuscular volume, mean corpuscular hemoglobin, red cell distribution, mean platelet volume, lymphocytes, monocytes, and granulocytes were assessed at the beginning and post-intervention.

Data are represented as means±SEM. An independent sam-ples t test was used to compare demographic and baseline variable across the treatment group. For comparing among the four groups (placebo, intervention, before, and after) a one-way analysis of variance followed by Tukey's post-hoc test was employed. A p value of <0.05 was considered to be a statistically significant difference. For sleep and QOL data, a Mann-Whitney U-test was used to determine the difference between groups.

Results

A total of 46 male and female healthy volunteers (age: 25.8±12.1 years) were recruited for this study. Six subjects dropped out from this study, and a total of 19 subjects in the placebo and 21 subjects in the treatment completed the study over a period of 90 consecutive days.

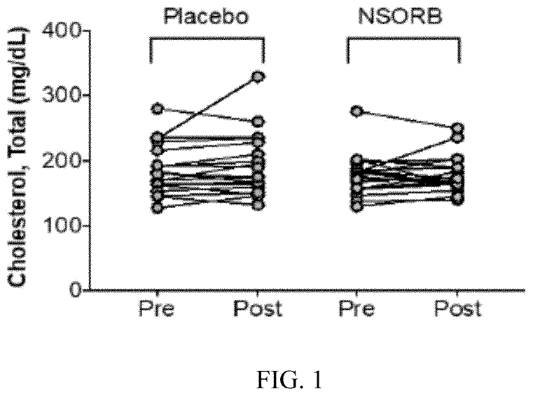

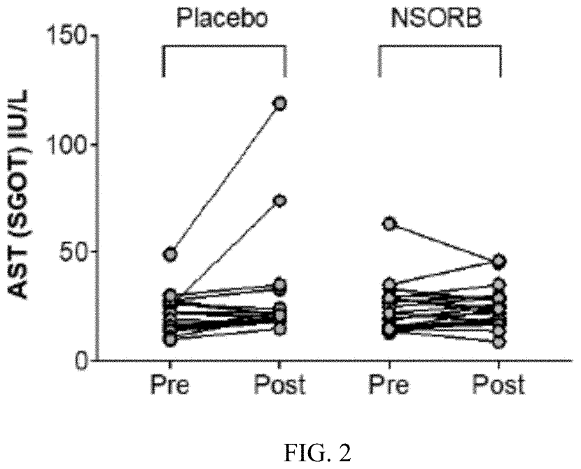

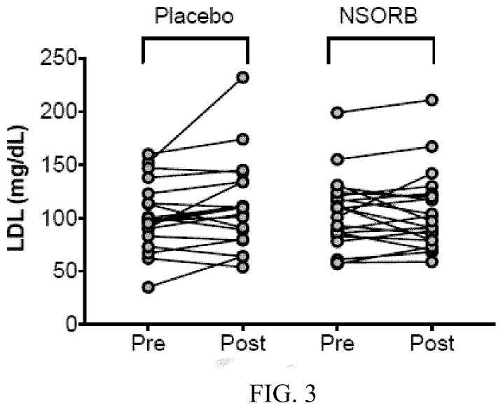

Detailed blood chemistry analyses before and after 90 days of intervention with N-SORB or placebo in healthy male and female human subjects are shown in Table 6 and FIGS. 1 through 4. No remarkable changes were observed. Total cholesterol levels increased in the placebo group following 90 days of treatment, while no such increase was observed in the N-SORB group (Table 6, FIG. 1). Of interest, the mean AST and ALT levels increased after 90 days of treatment in the placebo group (the reason for which is not known), while the group that received N-SORB evidenced a slight decreasing trend in AST levels (Table 6, FIG. 2). The HDL level in the placebo group was reduced following 90 days of treatment (Table 6). On the contrary, the HDL level did not change after 90 days of treatment in the N-SORB group (Table 6). The LDL level increased in the placebo group after 90 days of treatment; however, in the N-SORB-treated group, no increases were observed following 90 days of treatment (Table 6, FIG. 3).

Table 6 shows blood chemistry before and after 90 days of intervention with N-SORB or placebo in healthy human subjects

TABLE 6

| | Placebo | | N-SORB | |
|---|---|---|---|---|
| | Pre* | Post* | Pre* | Post* |
| BUN (mg/dL) | 13.8 ± 1.1 | 13.9 ± 0.9 | 14.8 ± 0.8 | 13.7 ± 3.3 |
| Creatinine, serum | 0.89 ± 0.04 | 0.86 ± 0.04 | 0.89 ± 0.03 | 0.84 ± 0.03 |
| Alkaline phosphatase (IU/L) | 68.4 ± 4.1 | 64.5 ± 3.9 | 64.24 ± 2.6 | 63.0 ± 2.6 |
| AST (SGOT) (IU/L) | 22.1 ± 2.1 | 30.2 ± 5.7^{#} | 24.5 ± 2.5 | 25 ± 0.9 |
| ALT (SGPT) (IU/L) | 23.1 ± 3.2 | 27.8 ± 6.8^{#} | 29.2 ± 3.3 | 28.7 ± 3.1 |
| Blood glucose (mg/dL) | 94.0 ± 14.5 | 92.8 ± 3.5 | 90.0 ± 1.9 | 89.7 ± 2.5 |
| Total cholesterol (g/dL) | 186 ± 8.8 | 193 ± 11 | 176 ± 6.9 | 178 ± 6.2 |
| Triglycerides (g/dL) | 117 ± 15.3 | 116 ± 14.4 | 115 ± 13.0 | 112 ± 13.7 |
| HDL (g/dL) | 57.4 ± 4.2 | 54.6 ± 3.9 | 52.2 ± 2.6 | 52.2 ± 3.0 |
| VLDL (g/dL) | 24.0 ± 3.0 | 23.1 ± 2.9 | 23.0 ± 2.6 | 23.9 ± 2.7 |
| LDL (g/dL) | 102 ± 7.4 | 112 ± 9.7 | 105 ± 7.3 | 105 ± 7.8 |

*Data are expressed as Mean ± SEM (placebo group = 19; N-SORB group = 21).
Pre-and post-values for placebo and N-SORB were treated as paired data and were compared by paired t-test.
No significant differences in the parameters tested were observed between pre-and post-data within the group following 90 days intervention
^{#}p < 0.01 compared to pre-treatment mean.
BUN-Blood Urea Nitrogen; Alkaline Phosphate-serum alkaline phosphatase; AST-Aspartate Aminotransferase; ALT-Alanine aminotransferase; HDL-High-density cholesterol; VLDL-Very low-density cholesterol; LDL-Low-density cholesterol.

Body composition analyses by dual-energy x-ray absorptiometry. Body composition analyses before and after 90 days intervention with N-SORB or placebo in healthy male and female human subjects are shown in Table 7. While no significant changes were observed in any of the parameters evaluated, a trend toward an increase in the BMI was observed in the group that received the placebo, which was not observed in the group that received N-SORB. Although these changes cannot be explained, it is likely that the eating habits during the winter season may have potentially contributed to the increase in BMI in the placebo group, or the N-SORB group may have some potential contribution in maintaining the BMI level.

TABLE 7

| | Placebo | | N-SORB | |
|---|---|---|---|---|
| | Pre* | Post* | Pre* | Post* |
| Tissue (% fat) | 23.4 ± 3.3 | 24.1 ± 5.2 | 29.1 ± 2.7 | 29.3 ± 2.8 |
| Region (% fat) | 27.2 ± 3.2 | 27.7 ± 3.4 | 31.4 ± 2.7 | 31.8 ± 2.7 |
| Tissue mass (g) | 72,345 ± 5,091 | 73,326 ± 5,042 | 77,163 ± 4,850 | 77,435 ± 4,863 |
| Fat mass (g) | 21,550 ± 3,725 | 22,276 ± 3,812 | 25,777 ± 2,944 | 26,197 ± 3,039 |
| Lean mass (g) | 50,836 ± 2,739 | 51,050 ± 2,731 | 51,449 ± 3,014 | 51,237 ± 3,052 |
| Android (% fat) | 33.7 ± 4.6 | 33.7 ± 4.6 | 37.9 ± 3.0 | 38.7 ± 3.0 |
| Gynoid (% fat) | 34.5 ± 3.3 | 34.9 ± 3.5 | 37.9 ± 3.0 | 38.7 ± 3.0 |
| Android/Gynoid | 0.989 ± 0.10 | 0.980 ± 0.10 | 0.983 ± 0.11 | 0.979 ± 0.07 |
| Body Mass Index | 24.74 ± 1.4 | 25.1 ± 1.6 | 27.63 ± 1.2 | 27.1 ± 1.8 |

*Data are expressed as Mean ± SEM (placebo group = 19; N-SORB group = 21).
Pre-and post-values for placebo and N-SORB were treated as paired data and were compared by paired t-test.
No significant differences in parameters tested were observed between pre-and post-data within the group following 90 days intervention.

Table 8 demonstrates the results of the total blood counts in the placebo and N-SORB treatment groups at the beginning of the study and at the end of 90 days of treatment in healthy human subjects. Parameters include white blood cells, red blood cells, hemoglobin, hematocrit, platelets, mean corpuscular volume, mean corpuscular hemoglobin, corpuscular hemoglobin concentration (CHC), red cell distribution, mean platelet volume, lymphocytes, monocytes, and granulocytes were assessed. There were not significant differences in the placebo or the intervention pre- or post-treatment in any of the parameters tested. The hematocrit level and mean platelet volume exhibited a decreasing trend in the placebo group, while a slight increase in these parameters was observed in the N-SORB group following 90 days of treat-ment. In contrast, platelet and % lymphocyte counts were lightly elevated in the placebo group after 90 days of treatment, while a decrease was observed in these parameters in the N-SORB group following 90 days of treatment.

TABLE 8

| | Placebo | | N-SORB | |
|---|---|---|---|---|
| | Pre* | Post* | Pre* | Post* |
| White Blood Cells (×10³ mm³) | 5.95 ± 1.7 | 6.1 ± 2.2 | 6.05 ± 1.8 | 6.13 ± 1.7 |
| Red Blood Cells (×10⁶ mm³) | 5.1 ± 0.5 | 4.8 ± 0.9 | 5.3 ± 0.5 | 5.2 ± 0.3 |
| Hemoglobin (g/dL) | 17 ± 4.5 | 18 ± 5.2 | 16 ± 3.5 | 16 ± 1.5 |
| Hematocrit (%) | 46 ± 5.1 | 44 ± 6.6 | 47 ± 4.2 | 49 ± 2.2 |
| Platelets (×10³ mm³) | 292 ± 84 | 312 ± 74 | 320 ± 94 | 301 ± 50 |
| Mean Corpuscular Volume (mm³) | 90 ± 8.3 | 86 ± 7.8 | 92 ± 8.3 | 96 ± 4.1 |
| Mean Corpuscular Hemoglobin (pg) | 30 ± 3.6 | 32 ± 4.4 | 30 ± 1.9 | 31 ± 2.6 |
| CHC (g/dL)** | 33 ± 1.4 | 33 ± 1.6 | 35 ± 2.6 | 34 ± 1.9 |
| Red cell distribution (%) | 15 ± 2.1 | 16 ± 3.3 | 15 ± 3.2 | 14 ± 2.4 |
| Mean Platelet Volume (μm³) | 7.0 ± 0.4 | 6.8 ± 0.8 | 6.9 ± 0.4 | 7.2 ± 0.8 |
| Lymphocytes (%) | 38 ± 9.7 | 41 ± 8.6 | 40 ± 8.4 | 35 ± 8.4 |
| Monocytes (%) | 5.3 ± 1.5 | 5.1 ± 1.1 | 4.8 ± 1.2 | 4.6 ± 1.4 |
| Granulocytes (%) | 57 ± 9.9 | 62 ± 8.5 | 60 ± 7.7 | 61 ± 8.4 |
| Lymphocytes # | 2.1 ± 0.5 | 2.0 ± 0.7 | 2.1 ± 0.5 | 2.1 ± 0.9 |
| Monocyte # | 0.3 ± 0.1 | 0.3 ± 0.2 | 0.2 ± 0.5 | 0.2 ± 0.1 |
| Granuloyte # | 4.5 ± 1.7 | 3.5 ± 1.3 | 3.3 ± 1.5 | 3.8 ± 1.2 |

*Data are expressed as Mean ± SEM (placebo group = 19; N-SORB group = 21).
**Corpuscular Hemoglobin Concentration.
***Pre-and post-values for placebo and N-SORB were treated as paired data and were compared by paired t-test. No significant differences in the parameters tested were observed between pre-and post-data within the group following 90 days intervention.

Blood Pressure and Cytokines

Detailed blood chemistry analyses before and after 90 days of intervention with N-SORB or placebo in healthy male and female human subjects are shown in Table 9. No remarkable changes were observed.

Total cholesterol levels increased in the placebo group following 90 days of treatment, while no such increase was observed in the N-SORB group (Table 6, FIG. 1). Of interest, the mean AST and ALT levels increased after 90 days of treatment in the placebo group (the reason for which is not known), while the group that received N-SORB did not show these changes (Table 6, FIG. 2). The HDL level in the placebo group was reduced following 90 days of treatment (Table 6). On the contrary, the HDL level did not change after 90 days of treatment in the N-SORB group (Table 6). The LDL level increased in the placebo group after 90 days of treatment; however, in the N-SORB-treated group, no increases were observed following 90 days of treatment (Table 6, FIG. 3).

TABLE 9

| | Placebo | | N-SORB | |
|---|---|---|---|---|
| | Pre* | Post* | Pre* | Post* |
| Blood pressure | | | | |
| (mm of Hg) | | | | |
| Systolic blood pressure | 122 ± 8 | 121 ± 9 | 122 ± 7 | 119 ± 11 |
| (SBP) | | | | |
| Diastolic blood pressure | 84 ± 7 | 82 ± 6 | 79 ± 7 | 83 ± 8 |
| (DBP) | | | | |
| Cytokines (pg/mL) | | | | |
| TNFα | 12.5 ± 8.2 | 14.6 ± 7.3 | 11.9 ± 6.2 | 12.1 ± 8.2 |
| IL-6 | 5.6 ± 3.0 | 4.3 ± 2.8 | 6.1 ± 2.9 | 4.6 ± 2.9 |
| IL-8 | 10.3 ± 4.4 | 14.6 ± 11 | 11.9 ± 9 | 12.1 ± 10 |

*Data are expressed as Mean ± SD (placebo group = 19; N-SORB group = 21).
Pre-and post-values for placebo and N-SORB were treated as paired data and were compared by paired t-test.
No significant differences in the parameters tested were observed between pre-and post-data within the group following 90 days intervention.
^{#}p < 0.01 compared to pre-treatment mean.
TNFα-Tumor necrosis factor alpha; IL-6-Interleukin 6; IL-8-Interleukin 8.

WHOQOL-BREF (QOL) and PSQI

Ratings for the five domains (overall quality, social relations, psychological health, environmental health, and physical health) measured by the WHOQOL-BREF, while the sleep pattern and sleep quality were assessed by PSQI (Table 10).

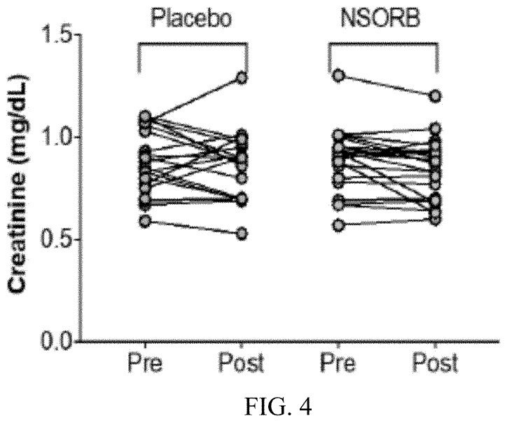

In the present study, physical health evaluation demonstrated some benefits in the N-SORB-treated subjects following 90 days of treatment (Table 10). A number of important benefits are (1) physical well-being, digestion, and sleep quality improved; (2) energy level increased; (3) reduced occurrence of headache and diarrhea; (4) cardiovascular health improved; (5) BMI reduced in N-SORB-treated subjects; (6) cholesterol level showed an increasing trend in the placebo group, while a lowering trend was observed in the N-SORB group (FIG. 1); (7) AST level showed an increasing trend in the placebo group, while a lowering trend was observed in the N-SORB group (FIG. 2); (8) LDL level showed an increasing trend in placebo group, while it remained stable in N-SORB group (FIG. 3); and (9) serum creatinine level showed an increasing trend in the placebo group, while a lowering trend was observed in the N-SORB group (FIG. 4).

Table 10 shows Pittsburgh Sleep Quality Assessment and Quality of Life Scores before and after 90 days of intervention with N-SORB or placebo in healthy human subjects

TABLE 10

| | Placebo | | N-SORB | |
|---|---|---|---|---|
| | Pre* | Post* | Pre* | Post* |
| PSQI Score | + | + | + | +++ |
| WHOQOL-BREF Score | + | + | + | +++ |

Pre-and post-Scores for placebo and N-SORB-treated subjects in the beginning and after 90-days of treatment.
PSQI-Pittsburg Sleep Quality Assessment (based on a set of nine questionnaire); WHOQOL-BREF-Quality of Life.

Digestion is the most important first step of metabolism, which is responsible for optimal health. As described earlier, enzymes naturally occurring in foods are intended to initiate the disintegration and dissolution of foodstuffs in the cardiac region of the stomach. However, food processing and high heat utilized for cooking denatures exogenous enzymes, rendering them nonfunctional, aborting disintegration of foodstuffs in the cardiac region of the stomach by food enzymes. Endogenous digestive enzyme secretions (primarily ptyalin) begin in the mouth by the salivary glands and have distinct functions in initiating the breakdown of ingested food material(s), primarily carbohydrates, into structurally diverse nutrients that facilitate their absorption in the body. Pancreatic enzyme secretions subsequently are intended to finish the process of disintegration and dissolution of foodstuffs. Different digestive enzymes have distinctive functions such as proteases and peptidases that disintegrate proteins into small peptides and amino acids; lipases break down fats into fatty acids and glycerol; amylases disintegrate complex carbohydrates into glucose and other monosaccharides; and nucleases break down nucleic acids into nucleotides.

However, the ability of the body to make endogenous enzymes for digestion and metabolism diminishes and wears out with nutritional deficiencies, excessive stress, digestive overburdening (i.e., overeating refined processed foods), advancing age, and various diseases. When digestion is impaired, it increases the demand for more discrete enzyme(s) for effective metabolism and digestion. A response to diminished enzyme production is a compensatory overproduction of digestive acid, which is well recognized as hyperacidity. The consequences of hyperacidity are acid reflux, heartburn, indigestion, diarrhea, and many other complications.

Conventional medical intervention for these complications include buffering antacids and proton pump inhibitors, which can temporarily relieve digestive discomfort but do not resolve the above problems caused by inadequate enzyme availability.

Endogenous digestive enzymes are site- and substrate-specific and mostly activated and work in a pH range of 2 to 5.5. N-SORB is a novel proprietary KD120 MEC metabolically activated enzyme formulation. These enzymes structurally resemble regular digestive enzymes such as amylase, protease, lipase, alpha galactosidase, and glucoamylase. However, N-SORB enzymes function in a pH range between 6.5 and 8.5. This specially engineered enzyme for-mulation utilizes an exclusive phospholipid (“Prodosome”) encapsulation technology that was shown to promote rapid and sustainable nutrient absorption of nutritional ingre-dients that effected positive changes in properties of the blood. Case studies, and preliminary observations in our laboratories have demonstrated that N-SORB-treated subjects had beneficial effects on their lifestyle. Subjects of different age group (age 20-71 years) have reported signifi-cant improvement of gastrointestinal functions, gut health, and increased metabolism without any adverse events. Other benefits include improved digestion, improvement of leaky gut syndrome with less gastrointestinal pain, less fatigue, less bloating, improved bowel movements, and increased energy level.

In the present investigation, a randomized, double-blind placebo-controlled study was conducted in 40 healthy male and female volunteers (mean age: 25.8±12.1 years) who consumed 1 mL of N-SORB or placebo, twice daily, over a period of 90 consecutive days. This project was designed to investigate the effects in healthy volunteers, especially as per the current Food and Drug Administration regulatory requirements. Physical health parameters were extensively monitored at the beginning and at the completion of the study. Several parameters including body weight, blood pres-sure, BMI, blood glucose, lipid profile, blood chemistry parameters, extensive body composition analyses, serum cytokine levels, WHOQOL-BREF, PSQI questionnaire, and extensive Quality of Life Questionnaire were completed at the beginning and end of the study (Table 10). Rigorous adverse events monitoring demonstrated broad spectrum safety of N-SORB supplementation.

Physical health parameters demonstrated that N-SORB treatment exhibited significant benefits. Physical well-being, sleep quality, energy levels, cardiovascular health, and overall lifestyle quality improved. Sleep quality is an integral part of good health. Also, sleep deprivation causes regular headache. Reduced occurrence of headache and fewer incidences of diarrhea were observed in the N-SORB-treated subjects. Furthermore, in contrast to the placebo group, BMI levels were marginally reduced in N-SORB-treated subjects, which clearly demonstrated the positive effect of N-SORB on lipid metabolism and lipid mobilization in these subjects. LDL level exhibited an increasing trend in the placebo group; however, it remained stable in the N-SORB group, which further confirmed the positive effect of N-SORB on lipid mobilization. Similarly, cholesterol levels exhibited a decreasing trend in the N-SORB group, while the placebo group showed an increasing trend at the end of 90 days (FIG. 1). Overall, these parameters further affirm the beneficial effects of N-SORB supplementation over a period of 90 consecutive days.

Several interesting observations were noted in the blood test profile panel following 90 days treatment with N-SORB. Hematocrit (% red blood cells in blood) represents the capability of the blood to deliver oxygen to the tissues. An increasing trend of the hematocrit level in the N-SORB group signifies a higher ability of the blood to transport oxygen, which is required and beneficial for optimal health (Table 8). It has been well demonstrated that the main function of platelets is to contribute to vascular healing and homeostasis. Thus, a decreasing trend of platelets in the N-SORB group is an indication that vascular endothelium gets stronger and healthier following treatment with N-SORB (Table 8). Conversely, an increase in mean platelet volume (MPV %), within the normal range, suggests larger and more effective platelets, which could result in a compensatory lower number of platelets. N-SORB treatment increased MPV % following a treatment of 90 consecutive days, while a decreasing trend was observed in the placebo group (Table 8), which is again a very positive sign.

Furthermore, a decreasing trend in mean corpuscular volume (MCV) in the placebo group was observed, while an increasing trend was observed in the N-SORB group over a treatment period of 90 consecutive days. It needs to be emphasized that a decreasing trend of MCV could be caused by kidney distress or failure. Decreased iron in the blood, anemia and other hemoglobinopathies can also cause a decrease in MCV. The ideal red cell distribution (%) lies between 11.5-14.5%, while a higher value indicates greater variation in size which is a hallmark of iron deficiency anemia. The red cell distribution (%) showed a decreasing trend leading to 14% in the N-SORB group, while the placebo group showed an increasing trend.

Lymphocytes, a subtypes of white blood cells, include natural killer cells, which function in a cell-mediated, cytotoxic innate immunity, T-cells for cell-mediated cytotoxic adaptive immunity and B-cells for humoral, antibody-driven adaptive immunity. N-SORB-treatment exhibited a lowering trend in lymphocyte %, which is again a healthy sign. In the placebo group there is an increasing trend. The increases in lymphocyte % have been demonstrated to be induced by acute viral infections, connective tissue diseases, thyrotoxicosis, adrenal exhaustion and splenomegaly with splenic sequestration of granulocytes.

Overall, the present randomized, double blind, placebo-controlled clinical study in forty healthy male and female subjects over a period of 90 consecutive days, demonstrated that N-SORB, a novel KD120 MEC enzyme formulation, improved the QOL, PSQI and selected parameters in young to middle age healthy volunteers, but didn't significantly alter cardiometabolic parameters, lipid profile or body composition. More clinical investigations in aging population are in progress to exhibit the beneficial effects of N-SORB in cardiometabolic parameters.

In conclusion, this randomized, double blind, placebo-controlled clinical study demonstrates that short-term intervention with N-SORB improves the QOL and PSQI in healthy volunteers and did not significantly alter cardiometabolic parameters, lipid profile, or body composition.

The use of the terms “a,” “an,” “the,” and similar referents in the context of describing the presently claimed invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value either above or below the stated value in a range of approximately ±5%; in other embodiments, the values may range in value either above or below the stated value in a range of approximately ±2%; in other embodiments, the values may range in value either above or below the stated value in a range of approximately ±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entireties. The present invention may be embodied in other specific forms without departing from the spirit or essential attributes thereof and, accordingly, reference should be made to the appended claims, rather than to the foregoing specification, as indicating the scope of the invention.

We claim:

1. A method for delivering an enzyme supplement, comprising the steps of:
(a) providing a formulation comprising at least one enzyme encapsulated in a multilamellar clustoidal phospholipid vehicle, the multilamellar clustoidal phospholipid vehicle comprising:
a solvent,
phosphatidylcholine of at least 70% purity, and
an ionic mineral composition; and
(b) orally administering the formulation to a human subject.

2. The method of claim 1, wherein the at least one enzyme is selected from the group consisting of a protease, a lipase, a carbohydrase, and an amylase.

3. The method of claim 1, wherein the at least one enzyme comprises the following Table:

| Ingredient | Source | Activity/Dose | mg |
|---|---|---|---|
| Amylase | Aspergillus oryzae | 10,309 DU | 68.7 |
| Protease | Aspergillus oryzae | 54,260 HUT | 67.8 |
| Lipase | Rhizopus oryzae | 1,107 FCCLU | 51.3 |
| Protease | Aspergillus oryzae | 24,010 HUT | 47.9 |
| Protease | Bacillus subtilis | 16,278 PC | 13.3 |
| Glucoamylase | Aspergillus niger | 13 AGU | 12.5 |
| Alpha-Galactosidase | Aspergillus niger | 54 GalU | 3.6 |
| Lipase | Aspergillus niger | 68 FCCLU | 3.3 |

4. The method of claim 3, wherein total cholesterol levels do not increase in the human subject 90 days after administering the formulation to the human subject.

5. The method of claim 3, wherein serum low density lipoprotein (LDL) levels do not increase in the human subject 90 days after administering the formulation to the human subject.

6. The method of claim 3, wherein serum high density lipoprotein (HDL) levels do not decrease in the human subject 90 days after administering the formulation to the human subject.

7. The method of claim 3, wherein sleep quality as measured by Pittsburgh Sleep Quality Assessment (PSQI) score is increased in the human subject 90 days after administering the formulation to the human subject in comparison with placebo.

8. The method of claim 3, wherein quality of life (QOL) as measured by World Health Organization Quality of Life (WHOQOL-BREF) score is increased in the human subject 90 days after administering the formulation to the human subject in comparison with placebo.

9. The method of claim 1, wherein the solvent is selected from the group consisting of water, an alcohol, and mixtures thereof.

10. The method of claim 1, wherein the ionic mineral composition comprises one or more of sodium ion, magnesium ion, chloride ion, potassium ion, sulfate ion, boron ion, lithium ion, phosphorous ion, manganese ion, calcium ion, silicon ion, selenium ion, zinc ion, iodine ion, chromium ion, copper ion, molybdenum ion, or vanadium ion.

11. The method of claim 1, wherein the phosphatidylcholine is soy lecithin phospholipid.

12. The method of claim 1, wherein the phosphatidylcholine is impregnated and saturated with the ionic mineral composition.

13. The method of claim 1, wherein the multilamellar clustoidal phospholipid vehicle is formulated in liquid dosage form.

14. The method of claim 1, wherein the multilamellar clustoidal phospholipid vehicle is formulated in solid dosage form.

15. The method of claim 1, wherein the ionic mineral composition is present in an amount from about 0.1 percent to about 12 percent by weight of the vehicle.

16. The method of claim 1, wherein the phosphatidylcholine is present in an amount from about 2 percent to about 20 percent by weight of the vehicle.

17. The method of claim 1, wherein the solvent is water present in an amount from about 40 percent to about 80 percent by volume of the vehicle.
